(19) United States
(12) Patent Application Publication
(10) Pub. No.: US 2015/0030506 A1
(43) Pub. Date: Jan. 29, 2015

(54) EMBEDDED MULTIFUNCTIONAL AIR PURIFIER USED IN KITCHEN

(71) Applicant: KOMPASS TECHNOLOGY CO., LTD., New Taipei City (TW)

(72) Inventors: CHING-CHENG KAO, NEW TAIPEI CITY 242 (TW); CHING-HUNG CHIEN, NEW TAIPEI CITY 242 (TW)

(21) Appl. No.: 14/513,405

(22) Filed: Oct. 14, 2014

Related U.S. Application Data
(63) Continuation-in-part of application No. 13/616,063, filed on Sep. 14, 2012.

(51) Int. Cl. A61L 9/16

(52) U.S. Cl. CPC A61L 9/16
USPC 422/121

(57) ABSTRACT

An embedded multifunctional air purifier for kitchen installed in a predetermined space of a cupboard includes a casing, a hood, an exhaust module, an electrostatic dust collector, a front filter, a gas detection module, a photocatalyst module and a vitamin C filter. With the design of the hood, the exhaust module is covered by the hood to reduce noises when the air purifier is used. Besides the removal of fumes and odors, filtration and disinfection, the air purifier can also produces fresh air with vitamin C, and alerts users to prevent a gas poisoning incident when a toxic gas leak occurs.

### CROSS-REFERENCE TO RELATED APPLICATIONS

This application is a continuation-in-part patent application of U.S. application Ser. No. 13/616,063 filed on Sep. 14, 2012, currently pending, the entire contents of which are hereby incorporated by reference for which priority is claimed under 35 U.S.C. §120.

### FIELD OF THE INVENTION

The present invention relates to the field of air purifiers, in particular to an embedded multifunctional air purifier for kitchen.

### BACKGROUND OF THE INVENTION

To reduce or eliminate fumes and smokes caused by cooking, various kinds of hood equipments are installed in kitchens, since the fumes produced in a kitchen contains high-risk carcinogenic substances including carbon monoxide, polycyclic aromatic smokes, heterocyclic amines, nitrated-polycyclic aromatic compounds, etc.

However, most conventional hood equipments can just filter the fumes only, and the effect of filtering the fume molecules is poor. In recent years, electrostatic dust removers are introduced to filter fumes and air and improve the effect of filtering fumes greatly, but these devices still cannot filter the fumes completely. In addition, there are other cooking utensils such as steaming stoves or ovens may produce various different odors in addition to the fumes. When these odors are mixed, the mixed odor may cause discomfort to the users. Therefore, embedded air purifiers for kitchen are introduced into the market, and products of this sort mainly adopt an electrostatic dust remover, a filter, an exhaust module, a photocatalyst module, an activated carbon filter, etc to remove remained fumes and odors produced by cooking and bacteria produced by leftovers in order to provide a comfortable air-conditioned kitchen.

However, the embedded air purifiers of this sort generally come with a compact size and a limited suction power, and thus affecting the air filtering effect of the kitchen. If an exhaust module with stronger suction and greater power is used, there will be an issue of loud noises. Based on the aforementioned reasons, the inventor of the present invention provides an embedded multifunctional air purifier for kitchen, and the air purifier with not only improves the suction and reduces the noises while maintaining its compact size, but also allows users to install it in the kitchen easily to remove fumes, odors and bacteria. In addition, the air purifier further includes a gas detector for detecting whether or not there is any toxic and harmful gas in the kitchen and warning users to avoid a gas poisoning incident and prevent the harmful gas from jeopardizing our health, so as to improve the safety of the kitchen substantially.

### SUMMARY OF THE INVENTION

In view of the drawbacks of the prior art, it is a primary objective of the present invention to provide a multifunctional air purifier capable of eliminating fumes and odors while supplying anions and releasing providing vitamin C particles in an air environment to enhance human immunity and prevent skin aging, so that kitchen users or housewives can have a healthy and safety environment when working in the kitchen for a long time, and the air purifier takes both suctions and noises into consideration, and provides the effect of installing, removing, maintaining and cleaning the air purifier conveniently.

Another objective of the present invention is to provide an embedded multifunctional air purifier for kitchen with a gas detection module for detecting and determining whether or not an inhaled gas is toxic to avoid the occurrence of gas poisoning caused by a gas leakage or preventing toxic gases from jeopardizing our health, so as to improve the safety of the kitchen substantially.

To achieve the aforementioned objective, the present invention provides an embedded multifunctional air purifier for kitchen embedded and installed in a predetermined space of a cupboard, and the embedded multifunctional air purifier comprises a casing with an air inlet and an air outlet, a gas channel formed in the casing, and the casing is provided for installing an electrostatic dust collector, an exhaust module, a front filter, a photocatalyst module and a vitamin C filter therein, wherein the front filter is installed at the air inlet, and the photocatalyst module is installed at a rear section of the gas channel, and the vitamin C filter is installed at the air outlet, and the embedded multifunctional air purifier is characterized in that a flip cover is covered onto the top of the casing, and the gas channel includes a hood installed therein, and the exhaust module is sealed and installed inside the hood, and the electrostatic dust collector is sealed and installed at an inlet of the hood, so that after the exhaust module sucks an airflow from the air inlet, the airflow is discharged from the air outlet through the gas channel, and a gas detection module is installed at the air inlet for detecting a status of toxicity of the airflow, and issuing a warning signal according to the status of toxicity.

In an embodiment, the embedded multifunctional air purifier for kitchen of the present invention further comprises a pair of fixed frames and a pair of slides, and the pair of fixed frames are installed on two opposite lateral sides of the casing respectively, and the pair of slides are fixed between the casing and the pair of fixed frames respectively, so that the casing is selectively pushed into the predetermined space and pulled out from the predetermined space by a sliding method to facilitate users to install, remove, maintain and clean the air purifier.

In addition, the exhaust module comprises a fixed board and a pair of fans, and the pair of fans are installed on the fixed board and adjacent to each other, and the fixed board is made with a cross-sectional shape corresponsive to the hood and sealed and installed inside the hood, wherein the shape of the hood can improve suctions and reduce noises significantly.

In addition, the gas detection module further comprises a warning lamp, a buzzer, a power disconnection unit and a notification unit, and after the warning lamp and the buzzer receive the warning signal, a warning with both sound and light effects is issued, and after the power disconnection unit receives the warning signal, the embedded multifunctional air purifier is automatically turned off, and after the notification unit receives the warning signal, a user and a firefighter are notified to avoid accidents.

Further, the photocatalyst module is comprised of a UV disinfection lamp and a photocatalyst filter and provided for eliminating bacteria and some viruses in the airflow.

In another embodiment, the embedded multifunctional air purifier for kitchen of the present invention further comprises a wireless receiver and a remote controller, electrically coupled to the embedded multifunctional air purifier, and the remote controller is provided for transmitting a wireless signal to the wireless receiver to turn on or off a power supply, so as to improve the convenience of use.

In another embodiment, the embedded multifunctional air purifier for kitchen of the present invention further comprises a magnetic acrylic board and a stainless steel panel, and the magnetic acrylic board are coupled and installed to a front side of the casing, and the stainless steel panel is fixed to the magnetic acrylic board by magnetic adhesion.

In a further embodiment, the embedded multifunctional air purifier for kitchen of the present invention further comprises an activated carbon filter installed at a rear section of the gas channel and disposed on a side of the photocatalyst module, for removing an odor.

### BRIEF DESCRIPTION OF THE DRAWINGS

FIG. 1 is a schematic view of installing an embedded multifunctional air purifier for kitchen in accordance with a preferred embodiment of the present invention;

FIG. 2 is a perspective view of an embedded multifunctional air purifier for kitchen in accordance with a preferred embodiment of the present invention;

FIG. 3 is an exploded view of an embedded multifunctional air purifier for kitchen in accordance with a preferred embodiment of the present invention; and

FIG. 4 is a schematic view of a gas flow occurred when using an embedded multifunctional air purifier for kitchen in accordance with a preferred embodiment of the present invention.

### DESCRIPTION OF THE PREFERRED EMBODIMENTS

The present invention will become clearer in light of the following detailed description of an illustrative embodiment of this invention described in connection with the drawings.

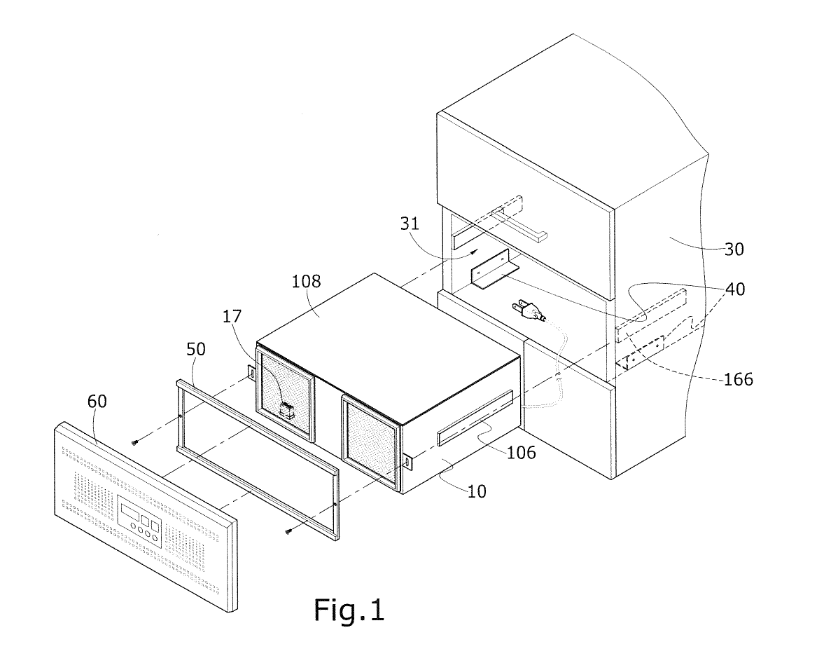

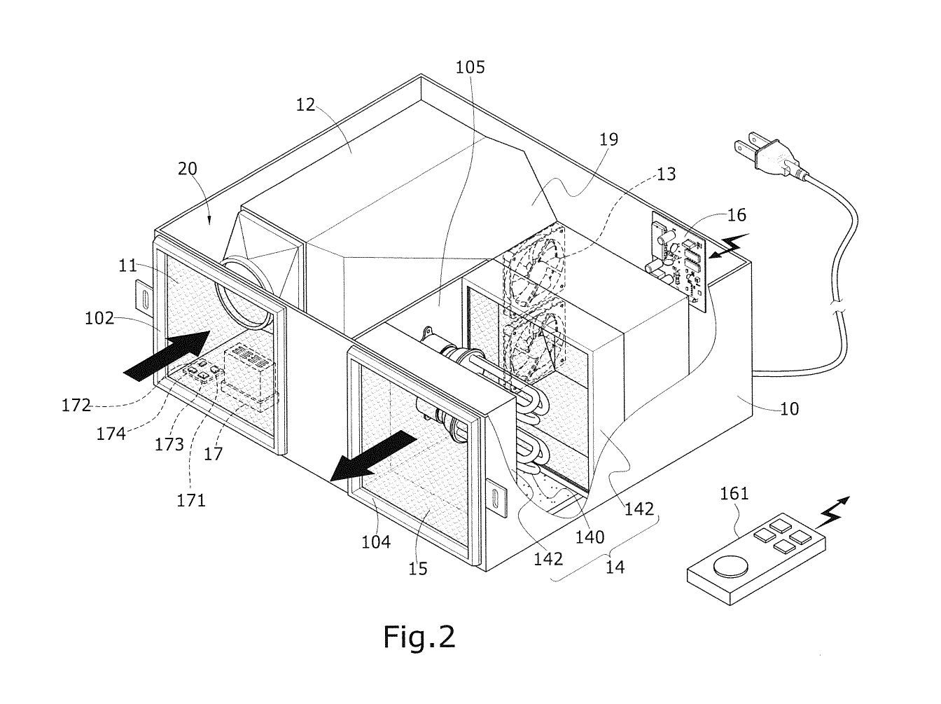

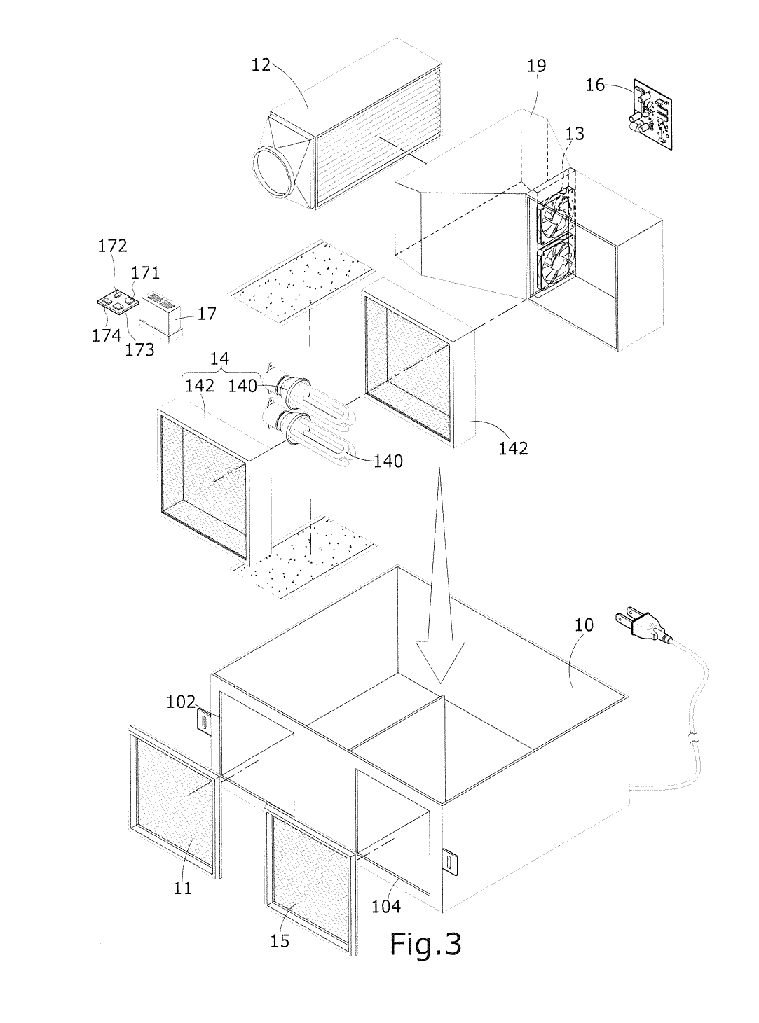

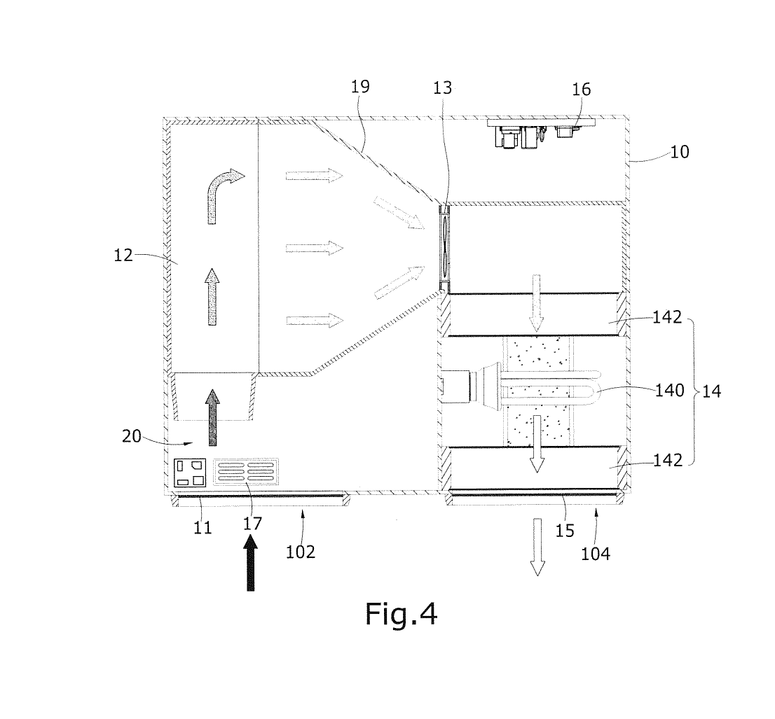

With reference to FIGS. 1 to 4 for a schematic view of installing an embedded multifunctional air purifier for kitchen, a perspective view and an exploded view of the air purifier, and a schematic view of a gas channel during the use of the air purifier in accordance with a preferred embodiment of the present invention respectively, the embedded multifunctional air purifier for kitchen 1 is installed a predetermined space 31 in of a cupboard 30, and the air purifier comprises a casing 10, a hood 19, an exhaust module 13, an electrostatic dust collector 12, a front filter 11, a gas detection module 17, a photocatalyst module 14 and a vitamin C filter 15. The casing 10 is a rectangular box structure, and after the casing 10 is embedded into a cupboard or a wall in a kitchen, a surface of the casing 10 in a directly contact with the kitchen environment comprises an air inlet 102 and an air outlet 104, a partition 105 installed between the air inlet 102 and the air outlet 104, and a gas channel 20 formed inside the casing 10, and both lateral sides adjacently coupled to a front side of the casing 10 have a slide 106 separately, and a section of the pair of slides 106 is fixed between the casing 10 and the predetermined space 31 by a pair of fixed frames 40, so that the casing 10 may be pushed into the predetermined space 31 or pulled out from the predetermined space 31 by a sliding method to produce a linear movement, so as to facilitate users to clean, maintain and replace the components of the air purifier.

The hood 19 is a funnel structure with a large inlet and a small outlet, and the inlet and the outlet are formed with angles perpendicular to each other and arranged inside the gas channel 20 to form a part of the gas channel 20. In addition, the electrostatic dust collector 12 is fixed to the inlet, and the inlet of the electrostatic dust collector 12 is parallel to the direction of the outlet of the hood 19 and corresponsive to the direction of the air inlet 102 and the air outlet 104, and such arrangement not just improves suctions and reduces noises effectively only, but also facilitate users to install, remove and maintain the air purifier.

In addition, the top of the casing 10 has a flip cover 108, so that when a user wants to maintain or clean the air purifier, the user pulls the casing 10 in the extending direction of the slide 106 and lifts the flip cover 108 to perform different operations quickly. Both ends of the gas channel 20 are coupled to the air inlet 102 and the air outlet 104 respectively. In other words, air particles in the kitchen environment are enter from the air inlet 102 into the casing 10, and then passed through the gas channel 20, and finally discharged from the air outlet 104 and back into the kitchen environment. When a gas flows through the gas channel 20, the gas passes through various different filters and purifiers such as the front filter 11, the electrostatic dust collector 12, the exhaust module 13, the photocatalyst module 14 and the vitamin C filter 15 or an activated carbon filter 18 installed at a rear section of the gas channel 20 for removing odors in the air. This circulation operation is repeated to obtain a cleaner air environment. In addition, the multifunctional air purifier of the present invention is embedded into a kitchen wall or a containing box, so that a user can control the operation of the air purifier more conveniently. The air purifier further comprises a wireless receiver 16 and a remote controller 161, both electrically coupled to the embedded multifunctional air purifier 1, and the remote controller 161 is provided for transmitting a wireless signal to the wireless receiver 16 to turn on or off a power supply.

An important characteristic of the present invention is to add a gas detection module 17 installed at the air inlet 102 and disposed on a side of the front filter 11 for detecting a status of toxicity of the airflow and issuing a warning signal according to the status of toxicity, and the gas detection module 17 further comprises a warning lamp 171, a buzzer 172, a power disconnection unit 173 and a notification unit 174. After the warning lamp 171 and the buzzer 172 receive the warning signal, a warning with sound and light effects is issued, and after the power disconnection unit 173 receives the warning signal, the embedded multifunctional air purifier for kitchen 1 is disconnected automatically, and after the notification unit 174 receives the warning signal, users and firefighters are notified.

It is noteworthy that the exhaust module 13 includes a fixed board 132 and a pair of fans 134, and the pair of fans 134 are arranged adjacent to each other on the fixed board 132 and installed inside the hood 19 to improve the amount of air, so as to improve the air intake and reduce the level of noise by approximately 2-3 dB.

In addition, the multifunctional air purifier of the present invention may be installed in a cupboard 30 or a kitchen wall, and a pair of fixed frames 40 are provided for supporting the casing 10 and fixing the casing 10 onto the fixed frames 40, and then a magnetic acrylic board 50 is attached onto surfaces of the air inlet 102 and the air outlet 104 of the multifunctional air purifier casing 10 embedded into the cupboard by a locking method or any other method, and finally a stainless steel panel 60 is attached onto the magnetic acrylic board 50 by magnetic attraction, so as to complete the installation procedure of the multifunctional air purifier.

In the air purification process, air particles in a kitchen are filtered by the front filter 11 installed at the air inlet 102, and the front filter 11 is provided for isolating larger dust particles in the air directly by a physical penetration method. To improve the isolation effect, the front filter 11 is a three-layer interlaced aluminum meshed structure capable of effectively preventing larger air particles to pass through. And then, the electrostatic dust collector 12 is installed at a front section of the gas channel 20 for passing the particles penetrating through the front filter 11 to an electric charge area, such that the air particles carry positive and negative charges. An electric field is provided for attaching and collecting larger particles to achieve the electrostatic dust collection effect. In particularly, the aforementioned technique can collect smaller dust mists and powder particles with a size of 0.2 microns.

To improve the air circulation efficiency, an exhaust module 13 is installed at a rear section of the gas channel 20 inside the casing 10 of the multifunctional air purifier for achieving the air exhaust effect, so as to expedite sucking the air particles in the kitchen into the air purifier and passing the flowing air particles through the front filter 11 and the electrostatic dust collector 12 into the exhaust module 13, and guiding the air particles into a next purification equipment. In the aforementioned purifying process, the photocatalyst module 14 is installed at a rear section of the gas channel 20, wherein the photocatalyst module 14 is comprised of a UV disinfection lamp 140 and a photocatalyst filter 142. After the photocatalyst is excited by ultraviolet light, peroxide molecules, superoxide molecules and strong oxide molecules with free radicals and having a strong reactivity are produced to decompose organic gases and odors into carbon dioxide and water. In addition, after microorganisms are in contact with a photocatalyst surface, the strong oxide molecules will destroy the outer membrane of the microorganisms and kill the microorganisms. Further, the photocatalyst filter 142 keeps degrading the attached gas molecules, and the cycle is repeated for re-adsorption and re-decomposition to achieve a permanent air purification effect. Unlike the general activated carbon which will lose the deodorant effect after the surface of the activated carbon has absorbed the odors up to a saturated level, the present invention does not require replacing the activated carbon to maintain the absorption function.

When the processed air particles are outputted from the air outlet 104, the processed air particles are passed through the vitamin C filter 15 installed at the air outlet 104, and then discharged from the vitamin C filter 15 to release vitamin C into the air, so as to achieve the effects of beautifying and moisturizing skins and inhibiting skin aging effectively.

In summation of the description above, the embedded multifunctional air purifier for kitchen of the present invention achieves the effects of isolating and collecting dusts, powders, fumes and dust mites in a kitchen, decomposing allergens and molds in the air to control and reduce the occurrence of allegoric diseases, and inhibiting and eliminating odors effectively. In addition, the air filter by the air purifier of the present invention and then discharged into kitchen environment further includes vitamin C to moisturize our skins and prevent skin aging. The invention provides a health and safety environment for kitchen users or housewives who usually work in a kitchen for a long time.

What is claimed is:

1. An embedded multifunctional air purifier for kitchen, embedded and installed in a predetermined space of a cupboard, and the embedded multifunctional air purifier comprising a casing with an air inlet and an air outlet, a gas channel formed in the casing, and the casing being provided for installing an electrostatic dust collector, an exhaust module, a front filter, a photocatalyst module and a vitamin C filter therein, wherein the front filter is installed at the air inlet, and the photocatalyst module is installed at a rear section of the gas channel, and the vitamin C filter is installed at the air outlet, and the embedded multifunctional air purifier is characterized in that a flip cover is covered onto the top of the casing, and the gas channel includes a hood installed therein, and the exhaust module is sealed and installed inside the hood, and the electrostatic dust collector is sealed and installed at an inlet of the hood, so that after the exhaust module sucks an airflow from the air inlet, the airflow is discharged from the air outlet through the gas channel, and a gas detection module is installed at the air inlet for detecting a status of toxicity of the airflow, and issuing a warning signal according to the status of toxicity.

2. The embedded multifunctional air purifier for kitchen according to claim 1, further comprising a pair of fixed frames and a pair of slides, and the pair of fixed frames being installed on two opposite lateral sides of the casing respectively, and the pair of slides are fixed between the casing and the pair of fixed frames respectively, so that the casing is selectively pushed into the predetermined space and pulled out from the predetermined space by a sliding method.

3. The embedded multifunctional air purifier for kitchen according to claim 2, wherein the exhaust module comprises a fixed board and a pair of fans, and the pair of fans are installed on the fixed board and adjacent to each other, and the fixed board is made with a cross-sectional shape corresponsive to the hood and sealed and installed inside the hood.

4. The embedded multifunctional air purifier for kitchen according to claim 3, wherein the gas detection module further comprises a warning lamp, a buzzer, a power disconnection unit and a notification unit, and after the warning lamp and the buzzer receive the warning signal, a warning with both sound and light effects is issued, and after the power disconnection unit receives the warning signal, the embedded multifunctional air purifier is automatically turned off, and after the notification unit receives the warning signal, a user and a firefighter are notified.

5. The embedded multifunctional air purifier for kitchen according to claim 4, wherein the photocatalyst module is comprised of a UV disinfection lamp and a photocatalyst filter.

6. The embedded multifunctional air purifier for kitchen according to claim 5, further comprising a wireless receiver and a remote controller, electrically coupled to the embedded multifunctional air purifier, and the remote controller being provided for transmitting a wireless signal to the wireless receiver to turn on or off a power supply.

7. The embedded multifunctional air purifier for kitchen according to claim 6, further comprising a magnetic acrylic board and a stainless steel panel, and the magnetic acrylic board being coupled and installed to a front side of the casing, and the stainless steel panel being fixed to the magnetic acrylic board by magnetic adhesion.

8. The embedded multifunctional air purifier for kitchen according to claim 7, further comprising an activated carbon filter installed at a rear section of the gas channel and disposed on a side of the photocatalyst module, for removing an odor.
